(19) United States
(12) Patent Application Publication
(10) Pub. No.: US 2004/0087613 A1
(43) Pub. Date: May 6, 2004

(54) Prevention of adhesions with rapamycin

(76) Inventor: Ernesto P. Molmenti, Baltimore, MD (US)

Correspondence Address: FRISHAUF, HOLTZ, GOODMAN & CHICK, P.C., ATTORNEY AT LAW, 767 THIRD AVENUE, NEW YORK, NY, 10017-2023, US

(21) Appl. No.: 10/349,904

(22) Filed: Jan. 23, 2003

Related U.S. Application Data
(60) Provisional application No. 60/352,541, filed on Jan. 29, 2002.

(51) Int. Cl. A61K 31/4745

(52) U.S. Cl. 514/291

(57) ABSTRACT

A method is provided for preventing or reducing the formation of adhesions or scar tissue either from the result of a surgical procedure or due to other irritation, trauma or other type of disruption of the tissue, by applying rapamycin or its derivatives or variants to a tissue.

A new use for rapamycin or a derivative or variant thereof, such as RAD, Certacan (Novartis Pharmaceutical Corporation) is provided, in particular to reduce or prevent adhesion and/or scar tissue.

Rapamycin is a macrocyclic triene antibiotic produced by Streptomyces hygroscopicus, which has been found to have many useful pharmaceutical properties including: antifungal activity, [U.S. Pat. Nos. 3,929,992 and 3,993,749]; and antitumor activity alone [U.S. Pat. No. 4,885,171] or in combination with picibanil [U.S. Pat. No. 4,401,653]. Rapamycin has also been found to be effective in both the experimental allergic encephalomyelitis model, a model for multiple sclerosis, and in the adjuvant arthritis model, a model for rheumatoid arthritis. And rapamycin has also been found to effectively inhibit the formation of IgE-like antibodies [R. Martel et al. [Can. J. Physiol. Pharmacol. 55, 48 (1977)]. The immunosuppressive effects of rapamycin have been disclosed in FASEB 3, 3411 (1989).

Rapamycin has also been shown to be useful in preventing or treating systemic lupus erythematosus [U.S. Pat. No. 5,078,999], pulmonary inflammation [U.S. Pat. No. 5,080,899], insulin dependent diabetes mellitus [Fifth Int. Conf. Inflamm. Res. Assoc. 121 (Abstract), (1990)], adult T-cell leukemia/lymphoma [European Patent Application 525,960 A1], and smooth muscle cell proliferation and intimal thickening following vascular injury [Morris, R. J. Heart Lung Transplant 11 (pt. 2): 197 (1992)].

Derivatives of rapamycin have been described in U.S. Reissued Pat. No. RE37,421 E (incorporated herein by reference) as being useful for inhibiting pathogenic fungi growth, immunosuppression or breast cancer. These derivatives can be used in the present invention.

The prior art, however, fails to teach the use of rapamycin to prevent or inhibit scar or adhesion formation in human or other mammalian patients.

One example of adhesions which are desired to be inhibited is Senechia (Senechiae), which is an adhesion that can occur inside the womb to fuse the walls of the womb together as a result of a number of different causes (sometimes known as Asherman's Syndrome). Causes include the surgical technique of dilatation and curettage (D & C), whether associated with induced abortion or not.

The use of rapamycin according to this invention may be indicated after the takedown of uterine scars or adhesions, after procedures in the uterus or adnexa that would lead to scar tissue formation, or in cases of infections that could cause scar formation. The use of rapamycin according to this invention can also be used in cases of Fallopian tube reconstructions.

Intra-uterine senechia can produce clinical symptoms such as menstrual abnormalities, infertility and habitual abortion (miscarriage). The occurrence of such complications appears to be on the rise.

The present invention includes the use of rapamycin or its derivatives or variants to help prevent apparent infertility in a woman caused by adhesions. (Successful treatment will alleviate other conditions as well.)

One usual way to treat adhesions is by surgery. That is, the adhered walls or tissues are separated surgically. The problem is that contact between the separated walls or tissues can allow the adhesions to reoccur.

The use of rapamycin, its derivatives or variants to the tissue can prevent adhesion or scar tissue formation.

It is therefore an object of the present invention to provide an improved method for alleviating the occurrence and reoccurrence of adhesions and scar tissue by locally or systemically applying rapamycin or its derivatives or variants to a tissue.

It is also an object to prevent or reduce the formation of keloids and/or to prevent other hypertrophic reactions by locally or systemically applying rapamycin or its derivatives or variants to a tissue.

### BRIEF DESCRIPTION

The present invention provides a method of preventing or reducing the formation of adhesions or scar tissue either from the result of a surgical procedure or due to other irritation, trauma or other type of disruption of the tissue, by applying rapamycin or its derivatives or variants to a tissue.

The present invention, for example provides an improvement to the surgical procedure for separating adhered tissue, e.g. the adhered walls of the womb, which improvement is the use of rapamycin a derivative or variant thereof to restrain or prevent the reformation of adhesions, by local or systemic administration.

For systemic administration, oral ingestion of dosages recommended for immunosuppressant purposes are usually about 2 mg one to three times a day. The problem with such oral administration, however, is that there will be a systemic immunosuppressant effect by the rapamycin. Such systemic effect may be unnecessary and may put a patient at risk associated with systemic immune suppression.

Therefore, the present invention also provides for local administration of rapamycin or a derivative or variant thereof either by subcutaneous injection or topical application via a pharmaceutically acceptable carrier.

For topical application, commercially available solutions of rapamycin which are in a concentration of 1 mg/ml can be used directly or diluted up to about 10% in pharmaceutically acceptable diluents.

Topical application can be performed by spraying or by dabbing or by other equivalent means.

### DETAILED DISCLOSURE

According to the present invention, rapamycin (sirolimus) or a derivative or variant thereof is systemically or locally applied as an agent to prevent the formation of scar tissue.

For example, rapamycin may be topcially applied, e.g. in the uterus, Fallopian (uterine) tubes and area around the ovaries. This use of rapamycin may be indicated after the takedown of uterine scars/adhesions, after procedures in the uterus or adnexa that would lead to scar tissue formation, or in cases of infections that could cause scar formation. It could also be used in cases of Fallopian tube reconstructions. In these cases, the rapamycin is utilized to improve fertility and prevent infertility.

Since rapamycin is a category C drug with respect to teratogenic effects and pregnancy, the timing of pregnancy needs is determined after a thorough conversation with the patient.

The dosage to be used varies according to the procedure being performed. A typical dosage for topical application of rapamycin is 1-3 mg/m² tissue surface area. Higher or lower levels can also be used.

Other uses include, without limit: prevention, especially after surgery, of intra-abdominal adhesions; chest/pleural adhesions, brain adhesions and corneal adhering to the eyelid after corneal operations or injuries. Also, the formation of scar tissue can be restrained or prevented e.g. on prosthetic valves in the heart or on pacemaker wires or endovascular prosthesis such as those used to repair aortic aneurysms.

In addition, rapamycin or a derivative or variant thereof may also be used according to the present invention to prevent the formation of keloids and/or to prevent other hypertrophic reactions.

Still further, rapamycin or a derivative or variant thereof may also be used to treat or prevent adhesions in the intestines, either within the intestines themselves or between the intestines and other organs, and to treat endometriosis and associated complications.

In each use, the rapamycin or derivative or variant thereof is either systemically or locally applied.

For systemic application, the rapamycin or derivative or variant thereof may be taken orally in an effective amount for a limited period of time in accordance with the symptoms to be treated. The limited period of time may be days, weeks, months or even several years.

For local application, the rapamycin or derivative or variant thereof may either be injected subcutaneously in an effective amount, or topically applied in an effective amount via a pharmaceutically acceptable carrier by spraying or dabbing or other similar means. In the case of topical application, the rapamycin or derivative or variant thereof may be applied in an amount of 1-3 mg/m² tissue surface area, although higher (e.g. 10 mg/m² or more) or lower levels of application are also effective.

Use in other mammals in addition to humans is also contemplated, such as to prevent or help in the treatment of Senechia in the eyelid of elephants in captivity, or to achieve the above described other utilities described above with respect to human patients.

I claim:

1. A method of preventing formation of adhesions on a tissue which comprises applying an effective amount of rapamycin or a derivative or variant thereof to the tissue.

2. The method according to claim 1, wherein the rapamycin or derivative or variant thereof is topically applied in a pharmaceutically acceptable carrier in a dosage of 1-3 mg/m² tissue surface area.

3. The method according to claim 2, wherein the rapamycin or derivative or variant thereof is applied by spraying or dabbing a solution thereof on the tissue.

4. The method according to claim 1, wherein the rapamycin or derivative or variant thereof is systemically applied for a limited period of time by oral ingestion.

5. A method of preventing formation of keloids and/or hypertrophic reactions which comprises applying an effective amount of rapamycin or a derivative or variant thereof to a tissue.
